(19) United States
(12) Patent Application Publication
(10) Pub. No.: US 2012/0039836 A1
(43) Pub. Date: Feb. 16, 2012

(54) HAIR COSMETIC

(76) Inventors: Tomonori Toyoda, Kanagawa (JP); Hideki Shimizu, Kanagawa (JP); Takumi Kurushima, Kanagawa (JP); Taizo Fujiyama, Kanagawa (JP); Kazuyuki Miyazawa, Kanagawa (JP)

(21) Appl. No.: 13/258,844

(22) PCT Filed: Apr. 2, 2010
(86) PCT No.: PCT/JP2010/056057

§ 371 Date: Oct. 28, 2011

(30) Foreign Application Priority Data
Apr. 6, 2009 (JP) 2009-092516

(51) Int. Cl. A61K 8/90; A61Q 5/00; A61Q 5/06

(52) U.S. Cl. 424/70.17

(57) ABSTRACT

Disclosed is a hair cosmetic which can achieve fixing of hair (hairstyle) by sticking and also has an excellent arranging ability (also including a restyling ability), and moreover has humidity resistance. Specifically disclosed is a hair cosmetic having a novel adhesive setting resin which is obtained by combining and polymerizing monomers having a specific structure and has a moderate firmness and a high adhesive force when forming a film and further comprising a setting agent resin. Accordingly, a hair cosmetic having both fixing ability and arranging ability, and also having excellent humidity resistance (hair set retaining ability) is obtained.

### CROSS REFERENCE TO RELATED APPLICATIONS

This application relates to and claims priority from PCT/JP2010/056057 filed Apr. 2, 2010, the entire contents of which are incorporated herein by reference, which in turn claims priority from JP 2009-092516 filed Apr. 6, 2009.

### FIGURE SELECTED FOR PUBLICATION

No Figures

### BACKGROUND OF THE INVENTION

1. Field of the Invention

The present invention relates to a hair cosmetic that has both the abilities to set and arrange hair (also including the ability to restyle hair) and is further excellent in moisture resistance. More specifically, the present invention relates to a hair cosmetic that is capable of setting hair based on fixation and restyling the hair and is excellent in the ability to retain set hairstyles, by virtue of a novel polymer contained therein and a setting resin further incorporated therein.

2. Description of the Related Art

Hair styling includes two functions: forming hairstyles and keeping the formed hairstyles. The principles on which these two functions are exerted are allegedly fixation and adhesion (Non-Patent Document 1).

Hair styling based on fixation is to set hair by forming a solid film using a film-forming agent (polymer resin) called a setting agent. For example, conventional hair gels or hair sprays are based mainly on a hair styling mechanism using a setting resin. For example, Patent Document 1 discloses a hair cosmetic mainly comprising a film-forming polymer such as polyvinylpyrrolidone, sodium polyacrylate, or a polyvinylpyrrolidone-polyvinyl acetate copolymer as a setting resin. Patent Document 2 discloses a hair cosmetic comprising a silylated urethane resin as a setting resin, and has reported that this hair cosmetic forms a film having both softness and hardness and has natural textures and a high ability to keep hairstyles.

However, the hair cosmetics obtained using the resins as described in Patent Documents 1 and 2 disadvantageously fails to restyle hair from a temporarily formed hairstyle due to a hard film formed by the setting resin and loses styling functions when the film is broken. Specifically, a problem of styling agents based on fixation brought about by such a setting resin is the poor ability to arrange hair, though they are excellent in setting hair.

On the other hand, styling based on adhesion is to allow hairs to adhere to one another by an oily ingredient. Hair liquids comprising an adhesive oily ingredient such as polyalkylene glycol as a main base, hair waxes that utilize the adhesiveness of a solid oil and have been preferred by the youth in recent years, and so on are known as such styling agents. For example, Patent Document 3 discloses a cosmetic for hair that comprises waxes and a spinnable water-soluble polymer and is excellent in restyling hair.

However, hair styling based on the adhesiveness of such an oily ingredient is characterized by being capable of restyling through fingers or a brush because the oily ingredient retains flowability and adhesiveness on the hair. A problem of this hair styling is that the ability to set hair (ability to keep hairstyles) as in hair cosmetics comprising a setting resin is not obtained, though it is excellent in the so-called ability to arrange hair.

### PRIOR ART DOCUMENTS

### Non-Patent Documents

Non-Patent Document 1: “Development of Advanced Cosmetics II”, ed. by Masato Suzuki, published by CMC Publishing Co., Ltd., 1996, Chapter 10: Functions of Hair-Styling Agents and State-of-the-Art Technology

### Patent Documents

Patent Document 1: JP-A-2006-213706
Patent Document 2: JP-A-2003-171244
Patent Document 3: JP-A-Hei 10-45546

### ASPECTS AND SUMMARY OF THE INVENTION

### Problems to be solved by the Invention

Accordingly, an aspect of the present invention is to provide a hair cosmetic that is capable of setting hair (hairstyle) based on fixation, is also excellent in the ability to arrange hair (also including the ability to restyle hair), and further has the ability to retain set hairstyles enhanced by improving moisture resistance.

The present inventors have conducted diligent studies to attain the object and consequently completed the present invention by finding that: a hair cosmetic having both the abilities to set and arrange hair is obtained by incorporating, as a setting resin, a novel adhesive setting resin having moderate hardness and high adhesive strength during film formation; and moisture resistance (the ability to retain set hairstyles) can be improved by further incorporating therein a setting agent resin.

### Means for Solving the Problems

Specifically, the present invention provides

a hair cosmetic comprising an adhesive setting resin, a setting agent resin, and an alcohol, wherein the adhesive setting resin being obtained by polymerizing:
at least one monomer represented by the following formula (A) (hereinafter, referred to as a “monomer A”):

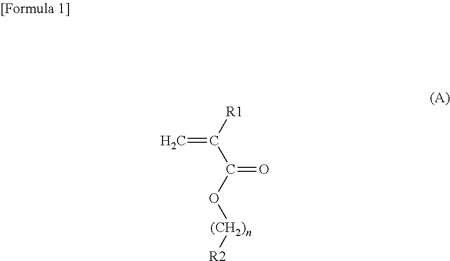

wherein R1 represents H or CH₃; n represents an integer of 0 to 30; (CH₂)ₙ contains a branched chain; and R2 represents H, OH, OCH₃, OCH₂CH₃, or phenyl,
and/or
at least one monomer represented by the following formula (B) (hereinafter, referred to as a “monomer B”):

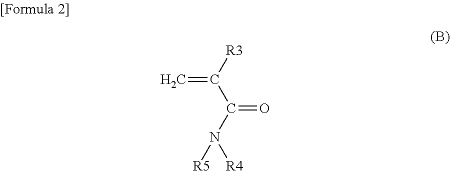

wherein R3 represents H or CH₃; R4 and R5, which may be the same or different, each represent H or (CH₂)₁R′; 1 represents an integer of 1 to 3; R′ represents H, OH, or —NR″R′″; and R″ and R′″, which may be the same or different, each represent H or a C1-C3 alkyl group,
and
at least one monomer represented by the following formula (C) (hereinafter, referred to as a “monomer C”):

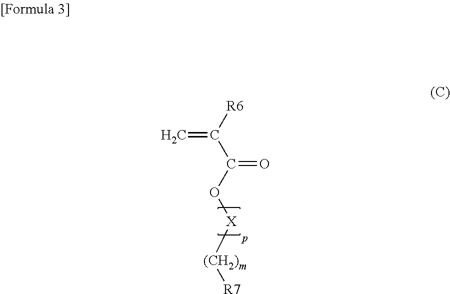

wherein R6 represents H or CH₃; p represents an integer of 1 to 100; m represents an integer of 0 to 30; R7 represents H, OH, OCH₃, OCH₂CH₃, or phenyl; and X represents an oxyethylene group (EO), an oxypropylene group (PO), an oxybutylene group (BO), or glyceryl,
and
at least one monomer represented by the following formula D (hereinafter, referred to as a “monomer D”):

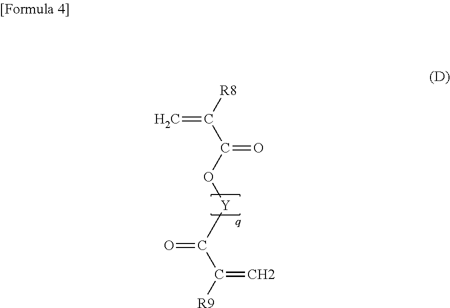

wherein R8 represents H or CH₃; q represents an integer of 1 to 100; and Y represents an oxyethylene group (EO), an oxypropylene group (PO), an oxybutylene group (BO), a linear or branched oxyalkylene group having 5 or more carbon atoms, or a glyceryl group, provided that q represents 1 when Y represents the oxyalkylene group having 5 or more carbon atoms.

### Effects of the Invention

A hair cosmetic of the present invention can have both the abilities to set and arrange hair, which are impossible to achieve for conventional setting resins, by incorporating therein the novel adhesive setting resin described above.

Furthermore, the hair cosmetic of the present invention can be excellent in moisture resistance and have the improved ability to retain set hairstyles for a long time, by further incorporating therein a setting agent resin conventionally used, in addition to the adhesive setting resin.

The above, and other aspects, features and advantages of the present invention will become apparent from the following description read in conjunction with the accompanying drawings, in which like reference numerals designate the same elements.

### BRIEF DESCRIPTION OF THE DRAWINGS

None

### DETAILED DESCRIPTION OF THE PREFERRED EMBODIMENTS

### Modes for Carrying Out the Invention

A hair cosmetic of the present invention comprises, as an essential ingredient, an adhesive setting resin obtained by polymerizing the monomers A and/or B, and C, and D described above. Specifically, for the adhesive setting resin of the present invention, it is essential to comprise the monomers C and D. The adhesive setting resin of the present invention can comprise any one of the monomers A and B, or both. A resin (polymer) that lacks the monomer C or D produces neither favorable adhesive strength nor the ability to arrange hair (ability to restyle hair).

It is particularly preferred that the adhesive setting resin used in the present invention should have a structure represented by the following formula (I):

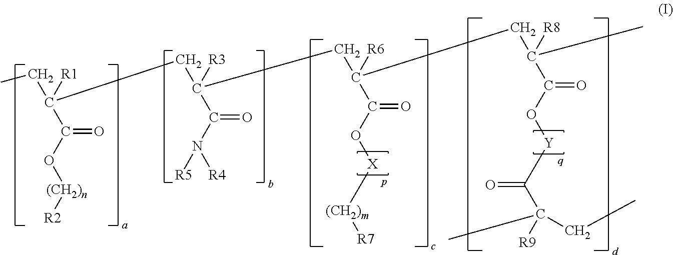

In the formula (I), R1 to R9, n, m, p, and q are as defined in the formulas A to D. The terms in the present specification are used as usual meanings. For example, an oxyethylene group (EO), an oxypropylene group (PO), and an oxybutylene group (BO) mean linear or branched oxyalkylene groups having 2, 3, and 4 carbon atoms, respectively. Moreover, in the formula (I), a represents a number within the range of 40<a<400; b represents a number within the range of 80≦b<300; c represents a number within the range of 30<c<300; and d represents a number within the range of 0<d<10.

The percentage by mass of each monomer in the adhesive setting resin (polymer of the formula (I)) that satisfies the conditions described above is approximately as follows: 7.5<A<62.5, 20≦B<45, 7.5<C<60, and 0<D<5.

The adhesive setting resin of the present invention can be prepared by mixing the monomers A and/or B, and C, and D at an appropriate ratio and polymerizing the mixture through reaction using a standard method, if necessary, in an appropriate solvent. For example, the adhesive setting resin can be obtained by thermally polymerizing the mixture at approximately 80° C. for 8 hours using a polymerization initiator such as 2,2′-azobisisobutyronitrile in ethanol. The obtained polymer can be purified appropriately for use.

The amount of the adhesive setting resin incorporated in the hair cosmetic of the present invention can vary depending on the product form thereof and is generally 0.1 to 30% by mass, preferably 1 to 20% by mass, more preferably 2 to 15% by mass. If the amount is less than 0.1% by mass, sufficient effect may not be exerted even in combination with a setting resin. If the adhesive setting resin is incorporated in an amount exceeding 30% by mass, the resulting hair cosmetic may make hair bristly.

The hair cosmetic of the present invention comprises a setting agent resin in addition to the adhesive setting resin. The setting agent resin in the present invention is an anionic, cationic, amphoteric, or nonionic film-forming polymer, also called a setting agent or a setting resin, or the like, and can be any of those conventionally incorporated in hair-styling agents. Specific examples thereof can include the followings:

### Acrylic and Vinyl Setting Agent Resins:

Anionic: alkyl acrylate/diacetoneacrylamide copolymers (PLASCIZE L-53P, PLASCIZE L-9909B, PLASCIZE L-9948B, etc. (all manufactured by GOO CHEMICAL CO., LTD.)), alkyl acrylate/octylacrylamide copolymers (Dermacryl 79 (manufactured by Nippon NSC Ltd.)), polyethylene glycol/polypropylene glycol-25/dimethicone/acrylate copolymers (LUVIFLEX SILK (manufactured by BASF)), acrylic acid/amide acrylate/ethyl acrylate copolymers (ULTRAHOLD 8 and ULTRAHOLD Strong (manufactured by BASF)), alkyl acrylate copolymers (ANISET NF-1000, ANISET HS-3000, etc. (manufactured by OSAKA ORGANIC CHEMICAL INDUSTRY LTD.)), etc.

Amphoteric: octylamide acrylate/hydroxypropyl acrylate/butylaminoethyl methacrylate copolymers (AMPHOMER SH30 and AMPHOMER LV-71 (manufactured by Nippon NSC Ltd.)), methacryloyloxyethylcarboxybetaine/alkyl methacrylate copolymers (YUKAFORMER R205, YUKAFORMER 301, YUKAFORMER SM, YUKAFORMER 104D, etc. (manufactured by Mitsubishi Chemical Corp.), and RAM Resin-1000, RAM Resin-2000, RAM Resin-3000, and RAM Resin-4000 (manufactured by OSAKA ORGANIC CHEMICAL INDUSTRY LTD.)), dimethyl diallyl ammonium chloride/acrylic acid copolymers (MERQUAT 280 and MERQUAT 295 (manufactured by Nalco Company)), dimethyl diallyl ammonium chloride/acrylamide/acrylic acid copolymers (MERQUAT Plus 3330 and MERQUAT Plus 3331 (manufactured by Nalco Company)), etc.

Cationic: vinylpyrrolidone/dimethylaminoethyl methacrylate copolymer diethyl sulfate (H.C. Polymer 1S (M) and H.C. Polymer 2 (manufactured by OSAKA ORGANIC CHEMICAL INDUSTRY LTD.), and GAFQUAT 755N (manufactured by ISP CORP.)), vinylpyrrolidone/dimethylaminopropylmethacrylamide/lauryl dimethylaminopropylmethacrylamide copolymers (STYLEZE W-20 (manufactured by ISP CORP.)), vinylpyrrolidone/N,N-dimethylamino ethyl methacrylate/alkyl acrylate/tripropylene glycol diacrylate copolymers (COSQUAT GA467 and COSQUAT GA468 (manufactured by OSAKA ORGANIC CHEMICAL INDUSTRY LTD.)), poly(dimethyl methylene piperidinium chloride) (MERQUAT 100 (manufactured by Nalco Company)), dimethyl diallyl ammonium chloride/acrylamide copolymers (MERQUAT 550 (manufactured by Nalco Company)), trimethylaminopropylacrylamide chloride/dimethylacrylamide copolymers, etc.

Nonionic: polyvinylpyrrolidone (LUVISKOL K17, LUVISKOL K30, and LUVISKOL K90 (manufactured by BASF), and PVP K (manufactured by ISP CORP.)), vinylpyrrolidone/vinyl acetate copolymers (PVP/VA S-630, PVP/VA E-735, and PVP/VA E-335 (all manufactured by ISP CORP.), LUVISKOL VA73W and LUVISKOL 37E (all manufactured by BASF), and PVA-6450 (manufactured by OSAKA ORGANIC CHEMICAL INDUSTRY LTD.)), vinylmethyl ether/alkyl maleate copolymers (GANTREZ A-425, GANTREZ ES-225, GANTREZ ES-335, etc. (manufactured by ISP CORP.)), vinylpyrrolidone/methacrylamide/vinylimidazole copolymers (LUVISET Clear (manufactured by BASF)), polyvinylcaprolactam (LUVISKOL Plus (manufactured by BASF)), etc.

### Urethane Setting Agent Resin:

IODOSOL PUD (manufactured by Nippon NSC Ltd.), LUVISET P.U.R. (manufactured by BASF), polymers described in JP-A-2006-213706, etc.; and acrylic urethane resins such as DynamX (manufactured by Nippon NSC Ltd.) and polymers described in Japanese Patent Application No. 2006-183144.

### Polysaccharide Setting Agent Resin:

Gum arabic, glucan, succinoglycan, carrageenan, karaya gum, tragacanth gum, guar gum, locust bean gum, galactomannan gum, xanthan gum, starch, carob gum, quince seed, casein, dextrin, gelatin, sodium pectate, sodium alginate, methylcellulose, ethylcellulose, carboxymethylcellulose, hydroxyethylcellulose, hydroxypropylcellulose, crystalline cellulose, O-[2-hydroxy-3-(trimethylammonio)propyl]hydroxyethylcellulose chloride, O-[2-hydroxy-3-(trimethylammonio)propyl]guar gum chloride, O-[2-hydroxy-3-(trimethylammonio)propyl]locust bean gum chloride, hydroxypropyl trimonium starch chloride, etc.

The setting agent resin incorporated in the hair cosmetic of the present invention is not particularly limited, and one or two or more of the setting agent resins as listed above can be selected appropriately for use. Acrylic, vinyl, or urethane setting agent resins are particularly preferable.

The amount of the setting agent resin incorporated in the hair cosmetic of the present invention is generally 0.1 to 30% by mass, preferably 0.5 to 20% by mass, more preferably 1 to 15% by mass. If the amount is less than 0.1% by mass, moisture resistance may be insufficient. If the setting agent resin is incorporated in an amount exceeding 30% by mass, the resulting hair cosmetic may make hair bristly.

The hair cosmetic of the present invention comprises an alcohol in addition to the adhesive setting resin and the setting agent resin.

One or two or more selected from alcohols generally used in cosmetics, such as ethanol, can be selected appropriately and used as the alcohol in the hair cosmetic of the present invention. The amount of the alcohol incorporated is not particularly limited and can vary depending on the form of the hair cosmetic. The alcohol is usually incorporated in an amount from the lower limit for use as a solvent in the adhesive setting resin to 80% by mass. Moreover, in some times, it is preferred that the amount of the alcohol incorporated should be adjusted according to the amount of water incorporated, in terms of controlling usability.

The hair cosmetic of the present invention comprises the novel adhesive setting resin and further comprises the setting agent resin. As a result, the hair cosmetic of the present invention exerts the abilities to set and arrange hair and exhibits excellent moisture resistance (ability to retain set hairstyles). Its form can be provided as various forms such as hair liquids, hair foams, hair mousse, hair sprays, hair mists, hair gels, and hair waxes.

The hair cosmetic of the present invention may comprise, for example, other ingredients conventionally used in hair cosmetics such as water according to the form thereof, without impairing the effect of the present invention.

### EXAMPLES

Hereinafter, the present invention will be described in more detail with reference to specific examples. However, the present invention is not intended to be limited to Examples below. Moreover, the amount of each ingredient incorporated in Examples, etc., below represents % by mass, unless otherwise specified.

### Production Examples and Comparative Production Examples

Monomers were polymerized according to the composition shown in Table 1 below to prepare adhesive setting resins of the present invention (Production Examples 1 to 6), a monomer C-free resin of Comparative Production Example 1, and a monomer D-free resin of Comparative Production Example 2.

Specifically, a mixture of monomers mixed in the total amount of 100 parts was prepared in advance. 100 parts of ethanol were added to a 1-L five-neck flask equipped with a dropping funnel containing this mixture, a reflux condenser, a thermometer, a tube for nitrogen substitution, and a stirrer. At the point in time when the ethanol was in a reflux state (approximately 80° C.) by heating under nitrogen flow, 1 part of a polymerization initiator (2,2′-azobisisobutyronitrile) was added into this ethanol, and the mixture was continuously added dropwise thereto for 2 hours. Then, the mixture was left for 8 hours in a reflux state to allow polymerization reaction to proceed. Next, the solvent was distilled off from the solution in the five-neck flask, and ethanol was added such that the solvent content in this solution was adjusted to obtain a hair cosmetic base solution having a 50% solid content.

TABLE 1

| | | | Pro- | Pro- | Pro- | Pro- | Pro- | Pro- | Com- | |
|---|---|---|---|---|---|---|---|---|---|---|
| | Chemical | | duction | duction | duction | duction | duction | duction | parative | Comparative |
| | structure | | Exam- | Exam- | Exam- | Exam- | Exam- | Exam- | Production | Production |
| Classification | (trade name) | Manufacturer | ple 1 | ple 2 | ple 3 | ple 4 | ple 5 | ple 6 | Example 1 | Example 2 |
| A | Butyl acrylate | Idemitsu | 40 | 35 | 30 | 15 | | | | 40 |
| | | Kosan Co., | | | | | | | | |
| | | Ltd. | | | | | | | | |
| A | Ethyl acrylate | TOAGOSEI | | | | | 30 | | 30 | |
| | | CO., LTD. | | | | | | | | |
| A | Stearyl | NOF CORP. | | | | | | | | |
| | methacrylate | | | | | | | | | |
| | (BLEMMER | | | | | | | | | |
| | SMA) | | | | | | | | | |
| A | Hydroxyethyl | OSAKA | | | | 15 | | | 30 | |
| | acrylate (HEA) | ORGANIC | | | | | | | | |
| | | CHEMICAL | | | | | | | | |
| | | INDUSTRY | | | | | | | | |
| | | LTD. | | | | | | | | |
| A | Methoxyethyl | TOAGOSEI | | | | | 15 | | | |
| | acrylate (Acrix | CO., LTD. | | | | | | | | |
| | C-1) | | | | | | | | | |
| B | Dimethylacrylamide | KOHJIN | | 40 | 30 | 30 | | 40 | 35 | 30 |
| | (DMAA) | CHEMICAL | | | | | | | | |
| | | CO., LTD. | | | | | | | | |
| B | Dimethylaminopropylacrylamide | TOAGOSEI | | | | | 20 | | | |
| | (DMAPAA) | CO., LTD. | | | | | | | | |
| C | Polyoxyethylene | NOF CORP. | 55 | 20 | 15 | 15 | | | | 30 |
| | glycol acrylate | | | | | | | | | |
| | (n = 10) | | | | | | | | | |
| | (BLEMMER | | | | | | | | | |
| | AE-400) | | | | | | | | | |
| C | Polyoxypropylene | NOF CORP. | | | 20 | 20 | 30 | 55 | | |
| | glycol acrylate | | | | | | | | | |
| | (n = 6) | | | | | | | | | |
| | (BLEMMER AP- | | | | | | | | | |
| | 400) | | | | | | | | | |
| D | Polyoxyethylene | Shin- | 5 | 5 | 5 | 5 | | | 5 | |
| | glycol diacrylate | Nakamura | | | | | | | | |
| | (n = 23) (NK | Chemical Co. | | | | | | | | |
| | ESTER A-1000) | | | | | | | | | |
| D | Polyoxyethylene | Shin- | | | | | | 5 | | |
| | glycol | Nakamura | | | | | | | | |
| | dimethacrylate | Chemical Co. | | | | | | | | |
| | (n = 14) (NK | | | | | | | | | |
| | ESTER 14G) | | | | | | | | | |
| D | Glycerin | NOF CORP. | | | | | 5 | | | |
| | dimethacrylate | | | | | | | | | |
| | (BLEMMER | | | | | | | | | |
| | NDMA) | | | | | | | | | |

### Examples and Comparative Examples

The resins of Production Examples and Comparative Production Examples were used to prepare samples. The samples were evaluated for the ability to arrange hair, the ability to set hair, the ability to restyle hair, and moisture resistance in use.

Evaluation methods and evaluation criteria for each property are shown below.

### 1. Ability to Arrange Hair

0.5 g of the sample was applied to one bundle of black virgin hair (length: 20 cm, mass: 2 g). After drying at room temperature, the hair bundle was evaluated for ease of arrangement in a sensory test by 10 female expert panelists.

### <Criteria of Evaluation Scores>

5: The hair was considerably easily arranged.
4: The hair was slightly easily arranged.

### 3: Normal.

2: The hair was not much easily arranged.
1: The hair was difficult to arrange.

### <Evaluation Criteria>

⊚: The total score was 40 or more.
◯: The total score was 30 or more and less than 40.
Δ: The total score was 20 or more and less than 30.
x: The total score was less than 20.

### 2. Ability to Set Hair

0.4 g of the sample was applied to black virgin hair (length: 15 cm, weight: 1 g), then spread over the hair using a comb, and styled such that the hair became straight. Five strands were prepared per sample. These strands were dried at 50° C. for 1 hour and then hung on a graduated board. A length (b) of each bent strand was measured in a thermo-hygrostat with a temperature of 30° C. and a humidity of 90% RH. The ability to set hair (ability to keep hairstyles) was determined according to a formula shown below using a length (a) of the bent strand measured in advance before application of the sample. A numeric value closer to 100% represents the higher ability to set hair and more excellent moisture resistance.

Ability to keep hairstyles (%)={(a−b)/a}×100

### <Evaluation Criteria>

⊚: The value was 90% or more.
◯: The value was 70% to less than 90%.
Δ: The value was 50% to less than 70%.
x: The value was less than 50%.

### 3. Ability to Restyle Hair

0.5 g of the sample was applied to one bundle of black virgin hair (length: 20 cm, mass: 2 g). After drying at room temperature, the hair bundle was evaluated for ease of restyling (ability to restyle hair) in a sensory test by 10 female expert panelists when the hair was styled immediately after the application and restyled one hour thereafter.

### <Criteria of Evaluation Scores>

5: Considerable ability to restyle hair.
4: Slight ability to restyle hair.

### 3: Normal.

2: Not much ability to restyle hair.
1: No ability to restyle hair.

### <Evaluation Criteria>

⊚: The total score was 40 or more.
◯: The total score was 30 or more and less than 40.
Δ: The total score was 20 or more and less than 30.
x: The total score was less than 20.

### 4. Moisture Resistance

0.5 g of the sample (hair cosmetic obtained in each Example or Comparative Example) was applied to black virgin hair (length: 20 cm, weight: 2 g). Immediately, the hair was curled using a curler having a curl diameter of 2 cm and dried at 50° C. for 1 hour. The length of a strand of this curled hair was measured and used as an initial value (L0).

Next, the dried hair bundle was hung on a graduated board and placed for 3 hours in a thermo-hygrostat with a temperature of 30° C. and a humidity of 90% RH. Then, the length of the hair strand was measured and used as a length (L2) after humidification.

The length of the hair strand is defined as the maximum diameter of curl in a curled state. On the other hand, it is defined as the maximum length from the end on the root side (e.g., the length from the end on the root side to the end on the hair tip side) when the hair is partially or wholly uncurled.

The curl retention value was calculated according to the following formula, and moisture resistance was evaluated according to the evaluation criteria shown below.

Curl retention value (%)={(20−L2)/(20−L0)}×100

A curl retention value closer to 100% represents the higher rate of curl retention and more excellent moisture resistance (i.e., ability to retain set hairstyles).

### <Evaluation Criteria>

⊚: The curl retention value was 90% or more.
◯: The curl retention value was 70% or more to less than 90%.
Δ: The curl retention value was 50% or more to less than 70%.
x: The curl retention value was less than 50%.

### (1) In the Case of Using Nonionic Resin as Setting Agent Resin

Samples were prepared according to the composition listed in Table 2 below. The samples were prepared by adding each polymer of (3) and/or (4) to a mixed solution of (1) and (2) and stirring the mixture. Subsequently, the properties of each sample were evaluated according to the criteria described above. The results are also shown in Table 2.

TABLE 2

| | Com- | Com- | | | | |
|---|---|---|---|---|---|---|
| | parative | parative | Ex- | Ex- | Ex- | |
| | Exam- | Exam- | am- | am- | am- | Exam- |
| | ple 1 | ple 2 | ple 1 | ple 2 | ple 3 | ple 4 |
| (1) Water | 60 | 60 | 59.9 | 59 | 55 | 50 |
| (2) Ethanol | 35 | 35 | 35 | 35 | 35 | 35 |
| (3) | 5 | | 5 | 5 | 5 | 5 |
| (Vinylpyrrolidone/VA) | | | | | | |
| copolymer | | | | | | |
| (4) Production | | 5 | 0.1 | 1 | 5 | 10 |
| Example 3 | | | | | | |
| Total | 100 | 100 | 100 | 100 | 100 | 100 |
| Ability to arrange hair | X | ◯ | Δ | Δ | ⊚ | ⊚ |
| Ability to set hair | Δ | Δ | Δ | Δ | ◯ | ◯ |
| Ability to restyle hair | X | ◯ | X | Δ | ◯ | ⊚ |
| Moisture resistance | Δ | Δ | ◯ | ◯ | ◯ | ◯ |

As is evident from the results shown in Table 2, the adhesive setting resin (Production Example 3)-free sample (Comparative Example 1) was inferior in the ability to arrange hair and the ability to restyle hair and also had insufficient moisture resistance. Moreover, the samples comprising both the adhesive setting resin and the setting agent resin (Examples 1 to 4) were improved in moisture resistance, compared with the sample free from the (vinylpyrrolidone/VA) copolymer as a setting agent resin (Comparative Example 2).

Samples were prepared according to the composition listed in Table 3 below, and the properties of each sample were evaluated according to the criteria described above. The results are also shown in Table 3.

TABLE 3

| | Comparative | Comparative | Comparative | | | | | | |
|---|---|---|---|---|---|---|---|---|---|
| | Example 1 | Example 3 | Example 4 | Example 5 | Example 6 | Example 3 | Example 7 | Example 8 | Example 9 |
| (1) Water | 60 | 55 | 55 | 55 | 55 | 55 | 55 | 55 | 55 |
| (2) Ethanol | 35 | 35 | 35 | 35 | 35 | 35 | 35 | 35 | 35 |
| (3) | 5 | 5 | 5 | 5 | 5 | 5 | 5 | 5 | 5 |
| (vinylpyrrolidone/ | | | | | | | | | |
| VA) | | | | | | | | | |
| copolymer | | | | | | | | | |
| (4) | | 5 | | | | | | | |
| Comparative | | | | | | | | | |
| Production | | | | | | | | | |
| Example 1 | | | | | | | | | |
| (4) | | | 5 | | | | | | |
| Comparative | | | | | | | | | |
| Production | | | | | | | | | |
| Example 2 | | | | | | | | | |
| (4) Production | | | | 5 | | | | | |
| Example 1 | | | | | | | | | |
| (4) Production | | | | | 5 | | | | |
| Example 2 | | | | | | | | | |
| (4) Production | | | | | | 5 | | | |
| Example 3 | | | | | | | | | |
| (4) Production | | | | | | | 5 | | |
| Example 4 | | | | | | | | | |
| (4) Production | | | | | | | | 5 | |
| Example 5 | | | | | | | | | |
| (4) Production | | | | | | | | | 5 |
| Example 6 | | | | | | | | | |
| Total | 100 | 100 | 100 | 100 | 100 | 100 | 100 | 100 | 100 |
| Ability to | X | Δ | Δ | ◯ | ⊚ | ⊚ | ⊚ | ◯ | ◯ |
| arrange hair | | | | | | | | | |
| Ability to set | Δ | ◯ | ◯ | Δ | ◯ | ◯ | ◯ | ◯ | Δ |
| hair | | | | | | | | | |
| Ability to | X | Δ | Δ | ◯ | ◯ | ◯ | ◯ | ◯ | ◯ |
| restyle hair | | | | | | | | | |
| Moisture | Δ | ◯ | Δ | ◯ | ◯ | ◯ | ◯ | ◯ | ◯ |
| resistance | | | | | | | | | |

The adhesive setting resin (Production Examples 1 to 6)-free sample (Comparative Example 1) and the samples (Comparative Examples 3 and 4) comprising the monomer C- or D-free resin (Comparative Production Example 1 or 2) failed to exert the sufficient abilities to arrange and restyle hair

### (2) In the Case of Using Anionic Resin as Setting Agent Resin

Samples were prepared according to the composition listed in Table 4 below. The samples were prepared by adding each polymer of (3) and/or (4) to a mixed solution of (1) and (2) and stirring the mixture. Subsequently, the properties of each sample were evaluated according to the criteria described above. The results are also shown in Table 4.

TABLE 4

| | Com- | Com- | Ex- | Ex- | | Ex- |
|---|---|---|---|---|---|---|
| | parative | parative | am- | am- | Exam- | am- |
| | Exam- | Exam- | ple | ple | ple | ple |
| | ple 5 | ple 2 | 10 | 11 | 12 | 13 |
| (1) Water | 60 | 60 | 59.9 | 59 | 55 | 50 |
| (2) Ethanol | 35 | 35 | 35 | 35 | 35 | 35 |
| (3) (Alkyl acrylate/ | 5 | | 5 | 5 | 5 | 5 |
| diacetoneacrylamide) | | | | | | |
| copolymer | | | | | | |
| (4) Production | | 5 | 0.1 | 1 | 5 | 10 |
| Example 3 | | | | | | |
| Total | 100 | 100 | 100 | 100 | 100 | 100 |
| Ability to arrange hair | X | ◯ | Δ | ◯ | ⊚ | ⊚ |
| Ability to set hair | ◯ | Δ | ◯ | ◯ | ◯ | ⊚ |
| Ability to restyle hair | X | ◯ | X | Δ | ◯ | ⊚ |
| Moisture resistance | ⊚ | Δ | ⊚ | ⊚ | ⊚ | ⊚ |

As is evident from the results shown in Table 4, the adhesive setting resin (Production Example 3)-free sample (Comparative Example 5) was inferior in the ability to arrange hair and the ability to restyle hair. The sample free from the (alkyl acrylate/diacetoneacrylamide) copolymer as a setting agent resin (Comparative Example 2) had insufficient moisture resistance.

Samples were prepared according to the composition listed in Table 5 below, and the properties of each sample were evaluated according to the criteria described above. The results are also shown in Table 5.

TABLE 5

| | Comparative | Comparative | Comparative | Example | Example | Example | Example | Example | Example |
|---|---|---|---|---|---|---|---|---|---|
| | Example 5 | Example 6 | Example 7 | 14 | 15 | 12 | 16 | 17 | 18 |
| (1) Water | 60 | 55 | 55 | 55 | 55 | 55 | 55 | 55 | 55 |
| (2) Ethanol | 35 | 35 | 35 | 35 | 35 | 35 | 35 | 35 | 35 |
| (3) (Alkyl | 5 | 5 | 5 | 5 | 5 | 5 | 5 | 5 | 5 |
| acrylate/ | | | | | | | | | |
| diacetoneacrylamide) | | | | | | | | | |
| copolymer | | | | | | | | | |
| (4) Comparative | | 5 | | | | | | | |
| Production | | | | | | | | | |
| Example 1 | | | | | | | | | |
| (4) Comparative | | | 5 | | | | | | |
| Production | | | | | | | | | |
| Example 2 | | | | | | | | | |
| (4) Production | | | | 5 | | | | | |
| Example 1 | | | | | | | | | |
| (4) Production | | | | | 5 | | | | |
| Example 2 | | | | | | | | | |
| (4) Production | | | | | | 5 | | | |
| Example 3 | | | | | | | | | |
| (4) Production | | | | | | | 5 | | |
| Example 4 | | | | | | | | | |
| (4) Production | | | | | | | | 5 | |
| Example 5 | | | | | | | | | |
| (4) Production | | | | | | | | | 5 |
| Example 6 | | | | | | | | | |
| Total | 100 | 100 | 100 | 100 | 100 | 100 | 100 | 100 | 100 |
| Ability to | X | Δ | Δ | ◯ | ⊚ | ⊚ | ⊚ | ◯ | ◯ |
| arrange hair | | | | | | | | | |
| Ability to set | ◯ | ◯ | ◯ | ◯ | ◯ | ◯ | ◯ | ◯ | ◯ |
| hair | | | | | | | | | |
| Ability to restyle | X | Δ | Δ | ◯ | ◯ | ◯ | ◯ | ◯ | ◯ |
| hair | | | | | | | | | |
| Moisture | ⊚ | ⊚ | ⊚ | ⊚ | ⊚ | ⊚ | ⊚ | ⊚ | ⊚ |
| resistance | | | | | | | | | |

The adhesive setting resin (Production Examples 1 to 6)-free sample (Comparative Example 5) and the samples (Comparative Examples 6 and 7) comprising the monomer C- or D-free resin (Comparative Production Example 1 or 2) failed to exert the sufficient abilities to arrange and restyle hair.

### (3) In the Case of Using Cationic Resin as Setting Agent Resin

Samples were prepared according to the composition listed in Table 6 below. The samples were prepared by adding each polymer of (3) and/or (4) to a mixed solution of (1) and (2) and stirring the mixture. Subsequently, the properties of each sample were evaluated according to the criteria described above. The results are also shown in Table 6.

TABLE 6

| | Compar- | Com- | Ex- | Ex- | Ex- | Ex- |
|---|---|---|---|---|---|---|
| | ative | parative | am- | am- | am- | am- |
| | Exam- | Exam- | ple | ple | ple | ple |
| | ple 8 | ple 2 | 19 | 20 | 21 | 22 |
| (1) Water | 60 | 60 | 59.9 | 59 | 55 | 50 |
| (2) Ethanol | 35 | 35 | 35 | 35 | 35 | 35 |
| (3) Polyquaternium-11 | 5 | | 5 | 5 | 5 | 5 |
| (4) Production | | 5 | 0.1 | 1 | 5 | 10 |
| Example 3 | | | | | | |
| Total | 100 | 100 | 100 | 100 | 100 | 100 |
| Ability to arrange hair | X | ◯ | Δ | ◯ | ⊚ | ⊚ |
| Ability to set hair | ◯ | Δ | ◯ | ◯ | ◯ | ⊚ |
| Ability to restyle hair | X | ◯ | X | Δ | ◯ | ⊚ |
| Moisture resistance | ◯ | Δ | ◯ | ◯ | ◯ | ◯ |

As is evident from the results shown in Table 6, the adhesive setting resin (Production Example 3)-free sample (Comparative Example 8) was inferior in the ability to arrange hair and the ability to restyle hair. The sample free from polyquaternium-11 as a setting agent resin (Comparative Example 2) had insufficient moisture resistance.

Samples were prepared according to the composition listed in Table 7 below, and the properties of each sample were evaluated according to the criteria described above. The results are also shown in Table 7.

TABLE 7

| | Comparative | Comparative | Comparative | Example | Example | Example | Example | Example | Example |
|---|---|---|---|---|---|---|---|---|---|
| | Example 8 | Example 9 | Example 10 | 23 | 24 | 21 | 25 | 26 | 27 |
| (1) Water | 60 | 55 | 55 | 55 | 55 | 55 | 55 | 55 | 55 |
| (2) Ethanol | 35 | 35 | 35 | 35 | 35 | 35 | 35 | 35 | 35 |
| (3) | 5 | 5 | 5 | 5 | 5 | 5 | 5 | 5 | 5 |
| Polyquaternium- | | | | | | | | | |
| 11 | | | | | | | | | |
| (4) Comparative | | 5 | | | | | | | |
| Production | | | | | | | | | |
| Example 1 | | | | | | | | | |
| (4) Comparative | | | 5 | | | | | | |
| Production | | | | | | | | | |
| Example 2 | | | | | | | | | |
| (4) Production | | | | 5 | | | | | |
| Example 1 | | | | | | | | | |
| (4) Production | | | | | 5 | | | | |
| Example 2 | | | | | | | | | |
| (4) Production | | | | | | 5 | | | |
| Example 3 | | | | | | | | | |
| (4) Production | | | | | | | 5 | | |
| Example 4 | | | | | | | | | |
| (4) Production | | | | | | | | 5 | |
| Example 5 | | | | | | | | | |
| (4) Production | | | | | | | | | 5 |
| Example 6 | | | | | | | | | |
| Total | 100 | 100 | 100 | 100 | 100 | 100 | 100 | 100 | 100 |
| Ability to | X | Δ | Δ | ◯ | ⊚ | ⊚ | ⊚ | ◯ | ◯ |
| arrange hair | | | | | | | | | |
| Ability to set hair | ◯ | ◯ | ◯ | ◯ | ◯ | ◯ | ◯ | ◯ | ◯ |
| Ability to restyle | X | Δ | Δ | ◯ | ◯ | ◯ | ◯ | ◯ | ◯ |
| hair | | | | | | | | | |
| Moisture | ◯ | ◯ | ◯ | ◯ | ◯ | ◯ | ◯ | ◯ | ◯ |
| resistance | | | | | | | | | |

The adhesive setting resin (Production Examples 1 to 6)-free sample (Comparative Example 8) and the samples (Comparative Examples 9 and 10) comprising the monomer C- or D-free resin (Comparative Production Example 1 or 2) failed to exert the sufficient abilities to arrange and restyle hair.

### (4) In the Case of Using Amphoteric Resin as Setting Agent Resin

Samples were prepared according to the composition listed in Table 8 below. The samples were prepared by adding each polymer of (3) and/or (4) to a mixed solution of (1) and (2) and stirring the mixture. Subsequently, the properties of each sample were evaluated according to the criteria described above. The results are also shown in Table 8.

TABLE 8

| | Com- | Com- | Ex- | Ex- | | Ex- |
|---|---|---|---|---|---|---|
| | parative | parative | am- | am- | Exam- | am- |
| | Exam- | Exam- | ple | ple | ple | ple |
| | ple 11 | ple 2 | 28 | 29 | 30 | 31 |
| (1) Water | 60 | 60 | 59.9 | 59 | 55 | 50 |
| (2) Ethanol | 35 | 35 | 35 | 35 | 35 | 35 |
| (3) | 5 | | 5 | 5 | 5 | 5 |
| (Methacryloyloxy- | | | | | | |
| ethylcarboxy | | | | | | |
| betaine/alkyl | | | | | | |
| methacrylate) | | | | | | |
| copolymer | | | | | | |
| (4) Production | | 5 | 0.1 | 1 | 5 | 10 |
| Example 3 | | | | | | |
| Total | 100 | 100 | 100 | 100 | 100 | 100 |
| Ability to arrange hair | X | ◯ | Δ | Δ | ⊚ | ⊚ |
| Ability to set hair | ◯ | Δ | ◯ | ◯ | ◯ | ◯ |
| Ability to restyle hair | X | ◯ | X | Δ | ◯ | ⊚ |
| Moisture resistance | Δ | Δ | ◯ | ◯ | ◯ | ◯ |

As is evident from the results shown in Table 8, the adhesive setting resin (Production Example 3)-free sample (Comparative Example 11) was inferior in the ability to arrange hair and the ability to restyle hair. The sample free from the (methacryloyloxyethylcarboxybetaine/alkyl methacrylate) copolymer as a setting agent resin (Comparative Example 2) had insufficient moisture resistance.

Samples were prepared according to the composition listed in Table 9 below, and the properties of each sample were evaluated according to the criteria described above. The results are also shown in Table 9.

TABLE 9

| | Comparative | Comparative | Comparative | Example | Example | Example | Example | Example | Example |
|---|---|---|---|---|---|---|---|---|---|
| | Example 11 | Example 12 | Example 13 | 32 | 33 | 30 | 34 | 35 | 36 |
| (1) Water | 60 | 55 | 55 | 55 | 55 | 55 | 55 | 55 | 55 |
| (2) Ethanol | 35 | 35 | 35 | 35 | 35 | 35 | 35 | 35 | 35 |
| (3) | 5 | 5 | 5 | 5 | 5 | 5 | 5 | 5 | 5 |
| (Methacryloyloxyethylcarboxy | | | | | | | | | |
| betaine/alkyl | | | | | | | | | |
| methacrylate) | | | | | | | | | |
| copolymer | | | | | | | | | |
| (4) Comparative | | 5 | | | | | | | |
| Production | | | | | | | | | |
| Example 1 | | | | | | | | | |
| (4) Comparative | | | 5 | | | | | | |
| Production | | | | | | | | | |
| Example 2 | | | | | | | | | |
| (4) Production | | | | 5 | | | | | |
| Example 1 | | | | | | | | | |
| (4) Production | | | | | 5 | | | | |
| Example 2 | | | | | | | | | |
| (4) Production | | | | | | 5 | | | |
| Example 3 | | | | | | | | | |
| (4) Production | | | | | | | 5 | | |
| Example 4 | | | | | | | | | |
| (4) Production | | | | | | | | 5 | |
| Example 5 | | | | | | | | | |
| (4) Production | | | | | | | | | 5 |
| Example 6 | | | | | | | | | |
| Total | 100 | 100 | 100 | 100 | 100 | 100 | 100 | 100 | 100 |
| Ability to | X | Δ | Δ | ◯ | ⊚ | ⊚ | ⊚ | ◯ | ◯ |
| arrange hair | | | | | | | | | |
| Ability to set | ◯ | ◯ | ◯ | ◯ | ◯ | ◯ | ◯ | ◯ | ◯ |
| hair | | | | | | | | | |
| Ability to | X | Δ | Δ | ◯ | ◯ | ◯ | ◯ | ◯ | ◯ |
| restyle hair | | | | | | | | | |
| Moisture | Δ | ◯ | ◯ | ◯ | ◯ | ◯ | ◯ | ◯ | ◯ |
| resistance | | | | | | | | | |

The adhesive setting resin (Production Examples 1 to 6)-free sample (Comparative Example 11) and the samples (Comparative Examples 12 and 13) comprising the monomer C- or D-free resin (Comparative Production Example 1 or 2) failed to exert the sufficient abilities to arrange and restyle hair.

### (5) In the Case of Urethane Resin as Setting Agent Resin

Samples were prepared according to the composition listed in Table 10 below. The samples were prepared by adding each polymer of (3) and/or (4) to a mixed solution of (1) and (2) and stirring the mixture. Subsequently, the properties of each sample were evaluated according to the criteria described above. The results are also shown in Table 10.

TABLE 10

| | Com- | Com- | | Ex- | Ex- | Ex- |
|---|---|---|---|---|---|---|
| | parative | parative | Exam- | am- | am- | am- |
| | Exam- | Exam- | ple | ple | ple | ple |
| | ple 14 | ple 2 | 37 | 38 | 39 | 40 |
| (1) Water | 60 | 60 | 59.9 | 59 | 55 | 50 |
| (2) Ethanol | 35 | 35 | 35 | 35 | 35 | 35 |
| (3) Silylated | 5 | | 5 | 5 | 5 | 5 |
| urethane resin*) | | | | | | |
| (4) Production | | 5 | 0.1 | 1 | 5 | 10 |
| Example 3 | | | | | | |
| Total | 100 | 100 | 100 | 100 | 100 | 100 |
| Ability to arrange hair | X | ◯ | Δ | ◯ | ⊚ | ⊚ |
| Ability to set hair | ⊚ | Δ | ⊚ | ⊚ | ⊚ | ⊚ |
| Ability to restyle hair | X | ◯ | X | Δ | ◯ | ⊚ |
| Moisture resistance | ⊚ | Δ | ⊚ | ⊚ | ⊚ | ⊚ |

*)Silylated urethane resin described as Production Example in JP-A-2006-213706.

As is evident from the results shown in Table 10, the adhesive setting resin (Production Example 3)-free sample (Comparative Example 14) was inferior in the ability to arrange hair and the ability to restyle hair. The sample free from the silylated urethane resin as a setting agent resin (Comparative Example 2) had insufficient moisture resistance.

Samples were prepared according to the composition listed in Table 11 below, and the properties of each sample were evaluated according to the criteria described above. The results are also shown in Table 11.

TABLE 11

| | Comparative | Comparative | Comparative | Example | Example | Example | Example | Example | Example |
|---|---|---|---|---|---|---|---|---|---|
| | Example 14 | Example 15 | Example 16 | 41 | 42 | 39 | 43 | 44 | 45 |
| (1) Water | 60 | 55 | 55 | 55 | 55 | 55 | 55 | 55 | 55 |
| (2) Ethanol | 35 | 35 | 35 | 35 | 35 | 35 | 35 | 35 | 35 |
| (3) Silylated | 5 | 5 | 5 | 5 | 5 | 5 | 5 | 5 | 5 |
| urethane | | | | | | | | | |
| resin*) | | | | | | | | | |
| (4) | | 5 | | | | | | | |
| Comparative | | | | | | | | | |
| Production | | | | | | | | | |
| Example 1 | | | | | | | | | |
| (4) | | | 5 | | | | | | |
| Comparative | | | | | | | | | |
| Production | | | | | | | | | |
| Example 2 | | | | | | | | | |
| (4) | | | | 5 | | | | | |
| Production | | | | | | | | | |
| Example 1 | | | | | | | | | |
| (4) | | | | | 5 | | | | |
| Production | | | | | | | | | |
| Example 2 | | | | | | | | | |
| (4) | | | | | | 5 | | | |
| Production | | | | | | | | | |
| Example 3 | | | | | | | | | |
| (4) | | | | | | | 5 | | |
| Production | | | | | | | | | |
| Example 4 | | | | | | | | | |
| (4) | | | | | | | | 5 | |
| Production | | | | | | | | | |
| Example 5 | | | | | | | | | |
| (4) | | | | | | | | | 5 |
| Production | | | | | | | | | |
| Example 6 | | | | | | | | | |
| Total | 100 | 100 | 100 | 100 | 100 | 100 | 100 | 100 | 100 |
| Ability to | X | Δ | Δ | ◯ | ⊚ | ⊚ | ⊚ | ◯ | ◯ |
| arrange hair | | | | | | | | | |
| Ability to set | ⊚ | ⊚ | ⊚ | ⊚ | ⊚ | ⊚ | ⊚ | ⊚ | ⊚ |
| hair | | | | | | | | | |
| Ability to | X | Δ | Δ | ◯ | ◯ | ◯ | ◯ | ◯ | ◯ |
| restyle hair | | | | | | | | | |
| Moisture | ⊚ | ⊚ | ⊚ | ⊚ | ⊚ | ⊚ | ⊚ | ⊚ | ⊚ |
| resistance | | | | | | | | | |

*)Silylated urethane resin described as Production Example in JP-A-2006-213706.

The sample (Comparative Example 14) comprising no adhesive setting resin (Production Examples 1 to 6) and the samples (Comparative Examples 15 and 16) comprising the monomer C- or D-free resin (Comparative Production Example 1 or 2) failed to exert the sufficient abilities to arrange and restyle hair.

The other Examples are shown below.

### Example 51

Liquid styling agent

| | | |
|---|---|---|
| (1) | ion-exchanged water | balance |
| (2) | PEG-6 | 5 |
| (3) | PEG-8 | 5 |
| (4) | PEG-32 | 5 |
| (5) | sorbitol | 3 |
| (6) | ethanol | 35 |
| (7) | fragrance | q.s. |
| (8) | polymer obtained in Production Example 3 | 5 |
| (9) | (alkyl acrylate/diacetone acrylamide) copolymer | 2.5 |
| (10) | citric acid | q.s. |

### <Production Process>

Water-soluble ingredients (2) to (5) were added to water (1) and dissolved by stirring to prepare aqueous parts. Next, (7) was added to (6), and the mixture was stirred for solubilization. Then, (8) and (9) were added thereto, and the mixture was stirred to prepare alcohol parts. The aqueous parts and the alcohol parts were mixed, and (10) was added to the mixture to obtain a liquid styling agent.

### Example 52

Hair-styling gel

| | | |
|---|---|---|
| (1) | carboxyvinyl polymer | 0.7 |
| (2) | polymer obtained in Production Example 3 | 5.0 |
| (3) | glycerin | 2.5 |
| (4) | 1,3-butylene glycol | 2.5 |
| (5) | polyoxyethylene octyldodecyl ether (20EO) | 0.5 |
| (6) | polyether-modified dimethylpolysiloxane | 1.0 |
| (7) | sodium hydroxide (adjusted to pH 7.5) | q.s. |
| (8) | ethanol | 20.0 |
| (9) | polyoxyethylene octyldodecyl ether | 0.1 |
| (10) | fragrance | 0.1 |
| (11) | trisodium edetate | 0.03 |
| (12) | ion-exchanged water | balance |
| (13) | (alkyl acrylate/diacetone acrylamide) copolymer | 5.0 |

### <Production Process>

(6) was added to (3), (4), (5), and a portion of (12), and the mixture was emulsified using a homo mixer. Subsequently, a portion of the remaining (12) was added to the emulsion to prepare emulsified parts. On the other hand, (1), (2), (7), (8), (9), (10), (11), and (13) were uniformly dissolved in the remaining portion of (12). To this solution, the emulsified parts were added to obtain a hair-styling gel emulsion.

### Example 53

Hair-styling gel

| | | |
|---|---|---|
| (1) | Hydroxyethylcellulose | 0.7 |
| (2) | Polymer obtained in Production Example 3 | 12.0 |
| (3) | Propylene glycol | 2.5 |
| (4) | 1,3-Butylene glycol | 2.5 |
| (5) | Polyoxyethylene hydrogenated castor oil (40EO) | 0.5 |
| (6) | Amino-modified high-molecular-weight silicone | 1.0 |
| (7) | Sodium hydroxide (adjusted to pH 7.5) | q.s. |
| (8) | Ethanol | 20.0 |
| (9) | Polyoxyethylene octyldodecyl ether | 0.1 |
| (10) | Fragrance | 0.1 |
| (11) | Trisodium edetate | 0.03 |
| (12) | Ion-exchanged water | balance |
| (13) | (Octylacrylamide/hydroxypropyl acrylate/ | 16.0 |
| | butylaminoethyl methacrylate) copolymer | |

### <Production Process>

A hair-styling gel was produced according to Example 52.

### Example 54

Hair-styling gel

| | | |
|---|---|---|
| (1) | (PEG-240/decyltetradeceth-20/HDI) copolymer | 2.0 |
| (2) | polymer obtained in Production Example 3 | 1.0 |
| (3) | diglycerin | 2.5 |
| (4) | polyethylene glycol 1000 | 2.5 |
| (5) | polyoxyethylene hydrogenated castor oil (40EO) | 0.5 |
| (6) | dimethylpolysiloxane modified with | 1.0 |
| | hydroxy at both ends (1,000,000 mPa · s) | |
| (7) | sodium hydroxide (adjusted to pH 7.5) | q.s. |
| (8) | ethanol | 20.0 |
| (9) | polyoxyethylene octyldodecyl ether | 0.1 |
| (10) | fragrance | 0.1 |
| (11) | trisodium edetate | 0.03 |
| (12) | ion-exchanged water | balance |
| (13) | (alkyl acrylate/diacetone acrylamide) copolymer | 1.0 |

### <Production Process>

A hair-styling gel was produced according to Example 52.

### Example 55

Hair-styling gel

| | | |
|---|---|---|
| (1) | Polymer obtained in Production Example 3 | 1.0 |
| (2) | Silylated urethane resin (Production | 2.5 |
| | Example of JP-A-2006-213706) | |
| (3) | Glycerin | 2.5 |
| (4) | Hydroxyethyl urea | 2.5 |
| (5) | Polyoxyethylene hydrogenated castor oil (40EO) | 0.5 |
| (6) | Dimethylpolysiloxane (1,000 mPa · s) | 1.0 |
| (7) | Triethanolamine (adjusted to pH 7.5) | q. s. |
| (8) | Ethanol | 20.0 |
| (9) | Polyoxyethylene octyldodecyl ether | 0.1 |
| (10) | Fragrance | 0.1 |
| (11) | Trisodium edetate | 0.03 |
| (12) | Ion-exchanged water | balance |
| (13) | (Octylacrylamide/hydroxypropyl acrylate/ | 1.0 |
| | butylaminoethyl methacrylate) copolymer | |

### <Production Process>

A hair-styling gel was produced according to Example 52.

### Example 56

Styling mousse

| | | |
|---|---|---|
| (1) | dimethylpolysiloxane (20 mPa · s) | 5.0 |
| (2) | isoparaffin | 5.0 |
| (3) | high-molecular-weight polysiloxane | 2.0 |
| (4) | amino-modified high-molecular-weight silicone | 0.5 |
| (5) | 1,3-butylene glycol | 3.0 |
| (6) | polyoxyethylene hydrogenated castor oil (40EO) | 2.0 |
| (7) | polymer obtained in Production Example 3 | 10.0 |
| (8) | polyoxyethylene/polyoxypropylene | 1.0 |
| | decyl ether (12EO•2PO) | |
| (9) | behenyl trimethyl ammonium chloride | 0.1 |
| (10) | phenoxyethanol | 0.1 |
| (11) | ethanol | 8.0 |
| (12) | ion-exchanged water | balance |
| (13) | fragrance | q.s. |
| (14) | (octylacrylamide/hydroxypropyl acrylate/ | 1.0 |
| | butylaminoethyl methacrylate) copolymer | |

### <Production Process>

(3) and (4) were dissolved in (1) and (2) by stirring, and this solution was added to (5), (6), and a portion of (12). The mixture was emulsified using a homo mixer (emulsified parts). On the other hand, (7) was added to the remaining portion of (12) (aqueous-phase parts). (8), (9), (10), (13), and (14) were added to (11) and dissolved by stirring. This solution was added to the aqueous-phase parts, to which the emulsified parts were further added and uniformly mixed to prepare a stock solution. 90 parts of this stock solution were charged into a can for aerosol. The can was valved, and 10 parts of liquefied petroleum gas (LPG) were charged thereto to obtain a styling mousse.

### Example 57

Styling mousse

| | | |
|---|---|---|
| (1) | Dimethylpolysiloxane (20 mPa · s) | 5.0 |
| (2) | High-molecular-weight methylpolysiloxane | 5.0 |
| | modified with hydroxy at both ends | |
| (3) | (Octylacrylamide/hydroxypropyl acrylate/ | 1.0 |
| | butylaminoethyl methacrylate) copolymer | |
| (4) | 1,3-Butylene glycol | 3.0 |
| (5) | Polyoxyethylene hydrogenated castor oil (40EO) | 2.0 |
| (6) | Polymer obtained in Production Example 3 | 0.0 |
| (7) | Polyoxyethylene cetyl ether (20EO) | 0.5 |
| (8) | Stearyl trimethyl ammonium chloride | 0.1 |
| (9) | Phenoxyethanol | 0.5 |
| (10) | Ethanol | 8.0 |
| (11) | Ion-exchanged water | balance |
| (12) | Fragrance | q.s. |

### <Production Process>

Styling mousse was produced according to Example 56.

### Example 58

Styling mousse

| | | |
|---|---|---|
| (1) | dimethylpolysiloxane (6 mPa · s) | 5.0 |
| (2) | isoparaffin | 5.0 |
| (3) | (PEG/amodimethicone) copolymer | 1.0 |
| (4) | 1,3-butylene glycol | 3.0 |
| (5) | polyoxyethylene hydrogenated castor oil (40EO) | 2.0 |
| (6) | polymer obtained in Production Example 3 | 1.0 |
| (7) | lauric acid diethanolamide | 0.2 |
| (8) | stearoxy hydroxypropylamine | 0.1 |
| (9) | phenoxyethanol | 0.1 |
| (10) | ethanol | 8.0 |
| (11) | ion-exchanged water | balance |
| (12) | fragrance | q. s. |
| (13) | (octylacrylamide/hydroxypropyl acrylate/ | 0.5 |
| | butylaminoethyl methacrylate) copolymer | |

### <Production Process>

A styling mousse was produced according to Example 56.

### Example 59

Styling mousse

| | | |
|---|---|---|
| (1) | Dimethylpolysiloxane (20 mPa · s) | 5.0 |
| (2) | Volatile isoparaffin | 5.0 |
| (3) | Polyether-modified high-molecular-weight silicone | 3.5 |
| (4) | 1,3-Butylene glycol | 3.0 |
| (5) | Polyoxyethylene hydrogenated castor oil (40EO) | 2.0 |
| (6) | Silylated urethane resin (Production Example | 10.0 |
| | of JP-A-2006-213706) | |
| (7) | Polyoxyethylene/polyoxypropylene behenyl | 1.0 |
| | ether (12EO•1PO) | |
| (8) | Cationized trehalose | 0.1 |
| (9) | Phenoxyethanol | 0.1 |
| (10) | Ethanol | 8.0 |
| (11) | Ion-exchanged water | balance |
| (12) | Fragrance | q.s. |
| (13) | Polymer obtained in Production Example 3 | 3.0 |

### <Production Process>

Styling mousse was produced according to Example 56.

### Example 60

Styling spray

| | | |
|---|---|---|
| (1) | Polymer obtained in Production Example 3 | 5.0 |
| (2) | Pyrocarboxylic acid-modified dimethylpolysiloxane | 0.5 |
| (3) | Alcohol | balance |
| (4) | Fragrance | q.s. |
| (5) | (Alkyl acrylate/diacetoneacrylamide) copolymer | 10.0 |

### <Production Process>

(1) to (5) were mixed and dissolved to prepare a stock solution. 50% of this stock solution and 50% of dimethyl ether were charged to an aerosol can to prepare an aerosol spray.

### Example 61

### Styling Spray

A stock solution was prepared in the same way as in Example 15. 99.33% of the stock solution and 0.67% of nitrogen gas were mixed and charged to an aerosol can to prepare an aerosol spray without the use of combustible gas.

### Example 62

Hair wax

| | | |
|---|---|---|
| (1) | kaolin | 1.0 |
| (2) | volatile isoparaffin | 5.0 |
| (3) | cetyl octanoate | 5.0 |
| (4) | phenylmethylpolysiloxane | 0.5 |
| (5) | candelilla wax | 3.0 |
| (6) | paraffin wax | 8.0 |
| (7) | propylene glycol | 5.0 |
| (8) | glyceryl stearate | 1.0 |
| (9) | polyoxyethylene glycerin monostearate (5EO) | 1.0 |
| (10) | isostearic acid | 1.0 |
| (11) | carboxyvinyl polymer | 0.4 |
| (12) | potassium hydroxide (adjusted to pH 7.5) | q.s. |
| (13) | ion-exchanged water | balance |
| (14) | polymer obtained in Production Example 3 | 5.0 |
| (15) | sorbitol | 3.0 |
| (16) | EDTA-2Na•2H₂0 | 0.05 |
| (17) | phenoxyethanol | 0.5 |
| (18) | fragrance | q.s. |
| (19) | highly polymerized polyethylene glycol | 0.1 |
| (20) | Silylated urethane resin | 1.0 |
| | (Production Example of JP-A-2006-213706) | |

### <Production Process>

(16), (7), and (15) were added to (13) and dissolved. Then, (11) was added to the solution and uniformly dispersed by stirring. (1) was added thereto and uniformly dispersed using Disper. The mixture was heated to 85° C., and a mixture of (2) to (9) dissolved by stirring at 85° C. in the same way as above was then added thereto and uniformly stirred. Then, (12) was added thereto, and the mixture was emulsified using a homo mixer. (14), (15), and (17) to (20) were sequentially added thereto, and the mixture was cooled to 25° C. to obtain a hair wax.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

Having described at least one of the preferred embodiments of the present invention with reference to the accompanying drawings, it is to be understood that the invention is not limited to those precise embodiments, and that various changes, modifications, and adaptations may be effected therein by one skilled in the art without departing from the scope or spirit of the invention as defined in the appended claims.

1. A hair cosmetic comprising:
an adhesive resin, a setting agent resin, and an alcohol, wherein:
the adhesive setting resin being obtained by polymerizing:
(A) at least one monomer represented by the following formula (A): 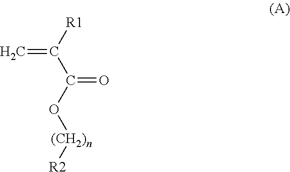
wherein R1 represents H or CH₃; n represents an integer of 0 to 30; (CH₂)ₙ contains a branched chain; and R2 represents one selected from the group consisting of H, OH, OCH₃, OCH₂CH₃, and phenyl, and
(B) at least one monomer represented by the following formula (B): 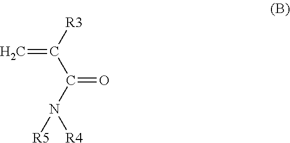
wherein R3 represents one of H and CH₃; R4 and R5 each represent at least one of a group consisting of H and (CH₂)₁R′; 1 represents an integer of 1 to 3; R′ represents one of a group consisting of H, OH, and —NR″R′″; and R″ and R′″ each represent one of H and a C1-C3 alkyl group, and
(C) at least one monomer represented by the following formula (C): 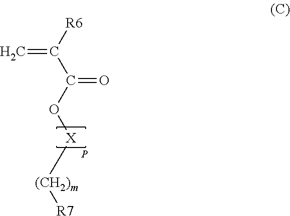
wherein R6 represents one of H and CH₃; p represents an integer of 1 to 100; m represents an integer of 0 to 30; R7 represents one selected from the group consisting of H, OH, OCH₃, OCH₂CH₃, and phenyl; and X represents one selected from the group consisting of an oxyethylene group (EO) an oxypropylene group (PO) an oxybutylene group (BO) and glyceryl, and
(D) at least one monomer represented by the following formula D: 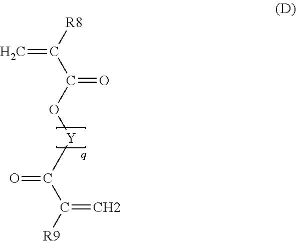
wherein R8 represents one of H and CH₃; q represents an integer of 1 to 100; and Y represents one selected from the group consisting of an oxyethylene group (EO), an oxypropylene group (PO), an oxybutylene group (BO), a linear or branched oxyalkylene group having 5 or more carbon atoms, and a glyceryl group, provided that q represents 1 when Y represents a linear or branched oxyalkylene group having 5 or more carbon atoms.

2. The hair cosmetic, according to claim 1, wherein:
the adhesive setting resin has a structure represented by the following formula (I): 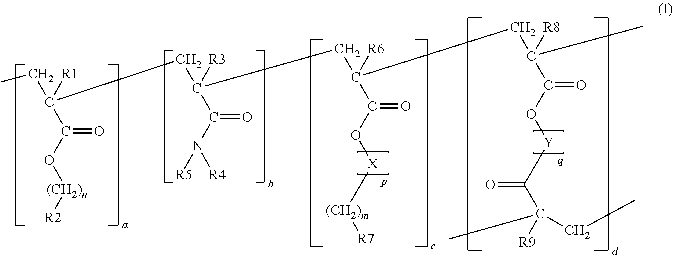
wherein R1 to R9, n, m, p, and q are as defined in the formulas A to D;
a represents a number within the range of 40<a<400;
b represents a number within the range of 80≦b<300;
c represents a number within the range of 30<c<300; and
d represents a number within the range of 0<d<10.

3. The hair cosmetic, according to claim 1, wherein:
the hair setting resin is at least one selected from the group consisting of a anionic, a cationic, an amphoteric, and a nonionic film-forming polymer.

4. The hair cosmetic, according to claim 2, wherein:
the hair setting resin is at least one selected from the group consisting of a anionic, a cationic, an amphoteric, and a nonionic film-forming polymer.
